SEQUENCE LISTING (8 sequences)

SEQ ID NO: 1 - DNA, 24 bp, synthetic construct
  source 1..24: mol_type = other DNA
cagtggtatggctgggaacaagac

SEQ ID NO: 2 - DNA, 24 bp, synthetic construct
  source 1..24: mol_type = other DNA
cagtggtatggctgggaacaagat

SEQ ID NO: 3 - DNA, 24 bp, synthetic construct
  source 1..24: mol_type = other DNA
aagttcgatgctctctccttagac

SEQ ID NO: 4 - DNA, 25 bp, synthetic construct
  source 1..25: mol_type = other DNA
aaaataaatcgtatcgccgggcagt

SEQ ID NO: 5 - DNA, 25 bp, synthetic construct
  source 1..25: mol_type = other DNA
aaaataaatcgtatcgccgggcagc

SEQ ID NO: 6 - DNA, 24 bp, synthetic construct
  source 1..24: mol_type = other DNA
aaaacacagagtatagccggttgg

SEQ ID NO: 7 - DNA, 301 bp, synthetic construct
  source 1..301: mol_type = other DNA
catggtaagtattaataattgtaaataagtttttttaaaagtttttttccactgttatattgaaaaaaaaacatagcaaggagttggtactcacacttgcaaaaccaaaacactgagccaaaatggccagtggtatggctgggaacaagayggagagttttgtccccaatgtaacctcctgcaaattcgccaaaaactgtctaaggagagagcatcgaactttggagacaagggtgaggtcagacttcttccgcagggaagaggatgacatgttgtgtgcggtccgaccggtccggttctc

SEQ ID NO: 8 - DNA, 301 bp, synthetic construct
  source 1..301: mol_type = other DNA
attagtatagccgtgtggcaatacttgatttttttttcaaattaatagtatagcaccgcggctatactattttttttcgattaatagtacagccacgcgctatactatttttttcactttttaaaaaaaataaatcgtatcgccgggcagytatactcctttttttaaaatattttaatgaaattaatagtatagccaaccggctatactctgtgttttattaatttttttaaaaaaattaatactagaattaatagtctagccgggcggctatactctgcacctttttgtttttaacgac

(19) United States
(12) Patent Application Publication
(10) Pub. No.: US 2026/0071284 A1
(43) Pub. Date: Mar. 12, 2026

(54) KOMPETITIVE ALLELE-SPECIFIC PCR (KASP) MOLECULAR MARKER PRIMER COMBINATION RELATED TO CHILLING REQUIREMENT (CR) OF PRUNUS PERSICA (L.) BATSCH, AND USE OF KASP MOLECULAR MARKER PRIMER COMBINATION

(71) Applicant: Jiangsu Academy of Agricultural Sciences, Nanjing (CN)

(72) Inventors: Juan YAN, Nanjing (CN); Zhixiang CAI, Nanjing (CN); Zheng CHEN, Nanjing (CN); Hongfeng SONG, Nanjing (CN); Lei GUO, Nanjing (CN); Binbin ZHANG, Nanjing (CN); Jianlan XU, Nanjing (CN); Zhijun SHEN, Nanjing (CN); Ruijuan MA, Nanjing (CN); Mingliang YU, Nanjing (CN)

(73) Assignee: Jiangsu Academy of Agricultural Sciences, Nanjing (CN)

(21) Appl. No.: 18/956,056

(22) Filed: Nov. 22, 2024

(30) Foreign Application Priority Data
Sep. 6, 2024 (CN) 202411248074.7
Oct. 14, 2024 (CN) 202411428600.8

(51) Int. Cl. C12Q 1/6895; C12Q 1/6811; C12Q 1/6858

(52) U.S. Cl. CPC C12Q 1/6895; C12Q 1/6858; C12Q 1/6811; C12Q 2600/13

(57) ABSTRACT

A Kompetitive allele-specific PCR (KASP) molecular marker primer combination related to a chilling requirement (CR) of Prunus persica (L.) Batsch and a use of the KASP molecular marker primer combination are provided. Based on a single-base difference between a base at a position 26,042,043 of chromosome 6 and a base at a position 46,470,090 of chromosome 1 for Prunus persica (L.) Batsch, a KASP molecular marker Chr06:26042043 primer combination and a KASP molecular marker Chr01:46470090 primer combination are designed. These two molecular marker primer combinations can be used to quickly and accurately identify a genotype of Prunus persica (L.) Batsch, such that a CR level of the Prunus persica (L.) Batsch can be preliminarily determined at a seedling stage. These two molecular marker primer combinations exhibit a high specificity and predictive ability in the detection of a CR of Prunus persica (L.) Batsch.

### CROSS REFERENCE TO THE RELATED APPLICATIONS

This application is based upon and claims priority to Chinese Patent Application No. 202411248074.7, filed on Sep. 6, 2024, and Chinese Patent Application No. 202411428600.8, filed on Oct. 14, 2024, the entire contents of which are incorporated herein by reference.

### SEQUENCE LISTING

The instant application contains a Sequence Listing which has been submitted in XML format via EFS-Web and is hereby incorporated by reference in its entirety. Said XML copy is named GBFD013-PKG_Sequence_Listing.xml, created on Nov. 21, 2024, and is 8,640 bytes in size.

### TECHNICAL FIELD

The present disclosure relates to a Kompetitive allele-specific PCR (KASP) molecular marker primer combination related to a chilling requirement (CR) of Prunus persica (L.) Batsch, and a use of the KASP molecular marker primer combination for assisting in screening of a Prunus persica (L.) Batsch variety with a CR level meeting a breeding need (a Prunus persica (L.) Batsch variety with a target CR). The present disclosure belongs to the field of molecular biology.

### BACKGROUND

Prunus persica (L.) Batsch is the third largest deciduous economic fruit tree worldwide and is mainly distributed in the temperate zone between 30°-45° north latitude and 30°-45° south latitude. During the winter dormancy period, Prunus persica (L.) Batsch requires a specified quantity of low-temperature exposure (namely, the CR) to promote the release of buds from endodormancy and the smooth flowering and leaf development. In recent years, the trend of global warming has been increasingly obvious, and abnormal climate phenomena such as warm winters, late spring coldness, and late frosts have occurred frequently, which brings new challenges to Prunus persica (L.) Batsch sensitive to temperature changes. Prunus persica (L.) Batsch with a high CR often cannot bloom and grow normally due to insufficient cold, and the planting region of Prunus persica (L.) Batsch, with a high CR, is constantly reduced, which also restricts the development of the peach industry towards the south. In addition, although Prunus persica (L.) Batsch with a low CR adapts to global warming and can be planted in the south, Prunus persica (L.) Batsch with a low CR is easily threatened by frosts due to the characteristics of early flowering and early leaf development. In contrast, Prunus persica (L.) Batsch with a high CR features late flowering and late leaf development and thus can avoid the harm of late spring coldness, but the high CR is not conducive to adaptation to global warming. In addition, the protected cultivation can bring excellent economic benefits, and in particular, an area of the protected cultivation in the north gradually expands. Therefore, there is an urgent need to accurately control the CR to improve the early control technologies. Therefore, the climate changes (including low-temperature deficiency and/or temperature abnormality) and industrial adjustments (accurate zoning cultivation, southward extension, protected cultivation, etc.) have put forward new requirements to ensure the sustainability of the peach industry. In order to ensure that Prunus persica (L.) Batsch can adapt to specific growth conditions and can be prevented from undergoing flowering and leaf development under non-optimal conditions, it is urgent to pay attention to the breeding of a target CR and comprehensively and quickly understand the CR level.

However, currently, the CR can only be identified through many years of observation after Prunus persica (L.) Batsch enters normal production, resulting in a low breeding efficiency. Therefore, it is necessary to achieve the early prediction of the CR with a modern molecular marker-assisted selection technique.

The marker-assisted breeding in fruit tree breeding relies on the identification of DNA patterns related to traits. The use of reference genomes, single nucleotide polymorphism (SNP) arrays, and genotyping-by-sequencing (GBS) for Prunus persica (L.) Batsch enhances the possibility of developing a cost-effective and user-friendly marker-assisted selection molecular tool. KASP has become a preferred method for developing an SNP-specific DNA test. KASP enables the rapid, cost-effective, and reliable determination of an SNP genotype. However, DNA tests for Prunus persica (L.) Batsch are still very limited and mostly involve the fruit quality or disease resistance. Based on the reported quantitative trait loci (QTLs) related to CRs, scholars have developed a high-resolution melting analysis-based genotyping toolkit to predict the CR. Recently, researchers in the United States have developed a KASP DNA assay with four CR-associated SNP markers in a CR-associated QTL segment identified on chromosome 1 of the genome for Prunus persica (L.) Batsch in previous studies. These molecular markers can distinguish among alleles for low, medium, and high CRs in Prunus persica (L.) Batsch breeding materials of Clemson University. In addition, Chinese scholars establish a PCR marker using an identified 30 bp deletion in the promoter of PpDAM6, a key gene for CRs. It has been verified through the local natural populations that this PCR marker can be used to distinguish between Prunus persica (L.) Batsch varieties with a CR of more than 500 h and a CR of less than 500 h.

These studies provide an important theoretical and methodological basis for the development of an efficient molecular tool targeting CR and marker-assisted breeding strategy for Prunus persica (L.) Batsch. However, the CR is a quantitative trait, and a variation of the quantitative trait may be explained by a plurality of loci together and is affected by environmental and population changes. The stability and validity of existing markers in specific environments and genetic groups can be well guaranteed. However, there remains a lack of stable and effective molecular markers for CRs of Prunus persica (L.) Batsch across large populations, complex genetic backgrounds, or varying environmental conditions. Therefore, the genomic prediction is required to correlate increased variations associated with the trait to ensure that a DNA test can accurately predict an actual phenotype and is applicable to a variety of genetic and environmental contexts, which provides a strong technical support for the selective breeding of Prunus persica (L.) Batsch with a target CR meeting various environmental and production requirements.

### SUMMARY

An objective of the present disclosure is to provide a KASP molecular marker primer combination related to a CR of Prunus persica (L.) Batsch and a use of the KASP molecular marker primer combination.

To achieve the above objective, the present disclosure adopts the following technical solutions:

The present disclosure discloses a KASP molecular marker Chr06:26042043 primer combination related to a CR of Prunus persica (L.) Batsch, including: a forward primer 1, a forward primer 2, and a shared reverse primer, where a nucleotide sequence of the forward primer 1 is shown in SEQ ID NO: 1, a nucleotide sequence of the forward primer 2 is shown in SEQ ID NO: 2, and a nucleotide sequence of the shared reverse primer is shown in SEQ ID NO: 3.

The present disclosure also discloses a KASP molecular marker Chr01:46470090 primer combination related to a CR of Prunus persica (L.) Batsch, including: a forward primer 1, a forward primer 2, and a shared reverse primer, where a nucleotide sequence of the forward primer 1 is shown in SEQ ID NO: 4, a nucleotide sequence of the forward primer 2 is shown in SEQ ID NO: 5, and a nucleotide sequence of the shared reverse primer is shown in SEQ ID NO: 6.

In order to distinguish between different genotypes, different fluorescent tags are added, respectively, to the 5′ terminus of the forward primer 1 and the forward primer 2 of each of the Chr06:26042043 primer combination and the Chr01:46470090 primer combination. Preferably, an FAM fluorescent tag (GAAGGTGACCAAGTTCATGCT) is added to a 5′ terminus of the forward primer 1, and a VIC fluorescent tag (GAAGGTCGGAGTCAACGGATT) is added to a 5′ terminus of the forward primer 2.

The present disclosure also discloses a use of the KASP molecular marker Chr06:26042043 primer combination or the KASP molecular marker Chr01:46470090 primer combination in the identification of a CR level of Prunus persica (L.) Batsch.

The CR level of Prunus persica (L.) Batsch refers to the classification of a CR of Prunus persica (L.) Batsch. Specifically, CRs of Prunus persica (L.) Batsch are classified as low, medium, high, and extremely high CRs, which correspond to CR<400 h, 400 h≤CR<600 h, 600 h≤CR<900 h, and CR≥900 h, respectively.

Preferably, the use includes the following steps:
(1) primer synthesis: synthesizing the KASP molecular marker Chr06:26042043 primer combination or the KASP molecular marker Chr01:46470090 primer combination;
(2) DNA extraction: extracting genomic DNA from the Prunus persica (L.) Batsch to be identified;
(3) KASP: with the genomic DNA extracted in the step (2) as a template, conducting the KASP using the KASP molecular marker Chr06:26042043 primer combination or the KASP molecular marker Chr01:46470090 primer combination synthesized in the step (1); and
(4) KASP product detection and analysis: detecting a genotype of an amplified product and preliminarily determining the CR level of the Prunus persica (L.) Batsch according to the genotype, where the step (1) and the step (2) are implemented in any order.

Preferably, a KASP system is a 5 μL PCR system including 2.5 μL of 2*KASP master mix, 1.25 μL of the primer combination, and 1.25 μL of the genomic DNA. A reaction procedure in step (3) is as follows: pre-denaturation at 95° C. for 10 min, 1 cycle; denaturation at 95° C. for 20 s; annealing-extension at 61° C. to 55° C. for 60 s, 10 cycles; denaturation at 95° C. for 20 s, and annealing-extension at 55° C. for 60 s, 27 cycles; and reading at 25° C. for 30 s, 1 cycle.

The present disclosure also discloses a combined use of molecular marker primer combinations in the identification of a CR level of Prunus persica (L.) Batsch, where the KASP molecular marker Chr06:26042043 primer combination and the KASP molecular marker Chr01:46470090 primer combination are used in combination to accurately identify the CR level of the Prunus persica (L.) Batsch.

The present disclosure also discloses a kit including the primer combination described above.

In the present disclosure, based on a single-base difference between a base at a position 26,042,043 of chromosome 6 and a base at a position 46,470,090 of chromosome 1 for Prunus persica (L.) Batsch, a KASP molecular marker Chr06:26042043 primer combination and a KASP molecular marker Chr01:46470090 primer combination are designed, respectively. In the molecular markers Chr06:26042043 and Chr01:46470090, an FAM fluorescent tag is added to a 5′ terminus of the forward primer 1, and a VIC fluorescent tag is added to a 5′ terminus of the forward primer 2. A genotype is determined according to a proportion of a fluorescence signal.

Prunus persica (L.) Batsch genotypes identified by the Chr06:26042043 marker include C:C, C:T, and T:T, respectively. Specifically, if a base C is detected at a locus, a proportion of an FAM fluorescence signal is high, and a genotype of Prunus persica (L.) Batsch to be identified is determined as a homozygous genotype C:C. If a base T is detected at the locus, a proportion of a VIC fluorescence signal is high, and a genotype of Prunus persica (L.) Batsch to be identified is determined as a homozygous genotype T:T. If both bases T and C are detected at the locus, that is, the proportions of FAM and VIC fluorescence signals are comparable, and a genotype of Prunus persica (L.) Batsch to be identified is determined as a heterozygous genotype C:T.

Prunus persica (L.) Batsch genotypes identified by the Chr01:46470090 marker include T:T, T:C, and C:C, respectively. Specifically, if a base T is detected at the SNP locus, a proportion of an FAM fluorescence signal is high, and a genotype of Prunus persica (L.) Batsch to be identified is determined as a homozygous genotype T:T. If a base C is detected at the locus, a proportion of a VIC fluorescence signal is high, and a genotype of Prunus persica (L.) Batsch to be identified is determined as a homozygous genotype C:C. If both bases T and C are detected at the locus, that is, the proportions of FAM and VIC fluorescence signals are comparable, and a genotype of Prunus persica (L.) Batsch to be identified is determined as a heterozygous genotype T:C.

The molecular markers of the present disclosure can be used to quickly and accurately identify a genotype of Prunus persica (L.) Batsch at the locus, so as to preliminarily determine a CR level of the Prunus persica (L.) Batsch. The present disclosure can identify a genotype of Prunus persica (L.) Batsch to preliminarily determine a CR level of the Prunus persica (L.) Batsch at a seedling stage quickly, which can reduce the time and labor costs and allow the high-throughput detection of a plurality of samples, thereby greatly improving detection efficiency. Thus, the present disclosure plays an important role in the selective breeding of Prunus persica (L.) Batsch with a target CR.

### BRIEF DESCRIPTION OF THE DRAWINGS

FIG. 1 shows the sequences and ideas for designing primers, where the sequence of bases from 26041893 to 26042193 of KASP molecular marker Chr06:26042043 is shown in SEQ ID NO: 7, and the sequence of bases from 46469940 to 46470240 of KASP molecular marker Chr01:46470090 is shown in SEQ ID NO: 8;

FIG. 2 is a schematic diagram of genotyping results of a KASP molecular marker Chr06:26042043 in Example 2;

FIG. 3 shows the CR distributions of different genotypes in Example 2;

FIG. 4 is a schematic diagram of genotyping results of a KASP molecular marker Chr01:46470090 in Example 3;

FIG. 5 shows the CR distributions of different genotypes in Example 3;

### DETAILED DESCRIPTION OF THE EMBODIMENTS

The specific implementations of the present disclosure are further described below in conjunction with the accompanying drawings, but the description of the embodiments does not limit the protection scope of the present disclosure.

Unless otherwise defined, all technical and scientific terms used herein have the same meanings as those generally understood by those skilled in the art of the present disclosure. The terms used in this specification of the present disclosure are only intended to describe the specific embodiments and are not intended to limit the present disclosure.

Unless otherwise specified, the materials or instruments used in the following examples can be acquired from the conventional commercial sources.

### Example 1 Design of KASP Molecular Marker Primer Combinations

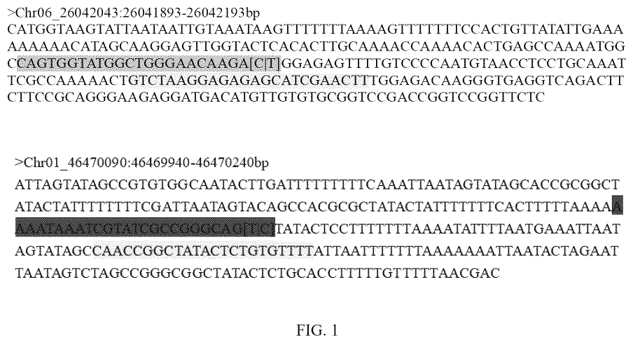

Upstream and downstream sequences >Chr06_26042043:26041893-26042193 bp and >Chr01_46470090:46469940-46470240 bp of SNP loci at 26,042,043 of chromosome 6 and 46,470,090 of chromosome 1 were searched, and KASP primers Chr06:26042043 and KASP primers Chr01:46470090 were designed with the Primer Premier 5.0 software to detect genotypes of Prunus persica (L.) Batsch with different CRs. The KASP primers Chr06:26042043 and the KASP primers Chr01:46470090 were designed in a 5′-3′ forward direction. Design ideas were shown in FIG. 1. Designed primer sequences were shown in Table 1. In FIG. 1, Chr06_26042043-forward primers (SEQ ID NO: 1 and SEQ ID NO: 2) were marked by a background, a Chr06_26042043-shared reverse primer (SEQ ID NO: 3) was marked by a background, Chr01:46470090-forward primers (SEQ ID NO: 4 and SEQ ID NO: 5) were marked by a background, and a Chr01:46470090-shared reverse primer (SEQ ID NO: 6) was marked by a background.

In order to distinguish different genotypes, an FAM fluorescent tag (GAAGGTGACCAAGTTCATGCT) was added to each of the 5′ terminus of a Chr06:26042043 forward primer 1 and a Chr01:46470090 forward primer 1, and a VIC fluorescent tag (GAAGGTCGGAGTCAACGGATT) was added to each of the 5′ terminus of a Chr06:26042043 forward primer 2 and a Chr01:46470090 forward primer 2. The primer combination sequences could specifically bind to corresponding sequences upstream and downstream of the SNP loci.

The KASP Chr06:26042043 primer combination and the KASP Chr01:46470090 primer combination were each prepared as follows: the respective forward primer 1, forward primer 2, and shared reverse primer were each diluted with Tris-EDTA (TE, pH 8.0) to 10 UM and then mixed according to a forward primer 1/forward primer 2/shared reverse primer ratio of 1:1:3, respectively.

TABLE 1
Sequences for KASP Chr06:26042043 and
Chr01:46470090 primers

| | |
|---|---|
| Chr06:26042043-forward | CAGTGGTATGGCTGGGAACAAGAC |
| primer 1 | (SEQ ID NO: 1) |
| Chr06:26042043-forward | CAGTGGTATGGCTGGGAACAAGAT |
| primer 2 | (SEQ ID NO: 2) |
| Chr06:26042043-shared | AAGTTCGATGCTCTCTCCTTAGAC |
| reverse primer | (SEQ ID NO: 3) |
| Chr01:46470090-forward | AAAATAAATCGTATCGCCGGGCAGT |
| primer 1 | (SEQ ID NO: 4) |
| Chr01:46470090-forward | AAAATAAATCGTATCGCCGGGCAGC |
| primer 2 | (SEQ ID NO: 5) |
| Chr01:46470090-shared | AAAACACAGAGTATAGCCGGTTGG |
| reverse primer | (SEQ ID NO: 6) |

### Example 2 Use of the KASP Molecular Marker Chr06:26042043

A total of 287 Prunus persica (L.) Batsch materials with rich genetic contexts (including cultivated varieties, local varieties, and wild resources from China, the United States, Thailand, etc., and belonging to germplasm types such as Prunus persica var. persica, Prunus kansuensis Rehder, Prunus davidiana, and Prunus ferganensis) were adopted as test materials. CRs of these test materials were determined (Table 2). 39 of the test materials had a low CR, 62 of the test materials had a medium CR, 164 of the test materials had a high CR, and 22 of the test materials had an extremely high CR. DNA was extracted from the 287 Prunus persica (L.) Batsch materials by a cetyltrimethylammonium bromide (CTAB) method, and the entire batch of DNA samples was diluted proportionally to a concentration of 1 ng/μL to 9 ng/μL.

The KASP Chr06:26042043 primers provided in Example 2 were used for genotyping of Prunus persica (L.) Batsch. A reaction system was as follows:
a 5 μL PCR system including 2.5 μL of 2*KASP master mix, 1.25 μL of the primer combination, and 1.25 μL of a diluted sample DNA (notes: in a negative control, the sample DNA was replaced with 1.25 μL of the buffer TE; and in a positive control, a Prunus persica (L.) Batsch material with a known loci SNP genotyping result produced based on first-generation sequencing (Sanger sequencing) was adopted (which was included in the above 287 Prunus persica (L.) Batsch materials, and included “Yuxia Pantao” with a C:C genotype and “Yejihong” with a T:T genotype). PCR analysis was conducted according to the instructions of the CFX Connect™ Real-Time System (Bio-Rad, USA) instrument. A PCR procedure was as follows: pre-denaturation at 95° C. for 10 min, 1 cycle; denaturation at 95° C. for 20 s; annealing-extension at 61° C. to 55° C. for 60 s, 10 cycles; denaturation at 95° C. for 20 s, and annealing-extension at 55° C. for 60 s, 27 cycles; and reading at 25° C. for 30 s, 1 cycle.

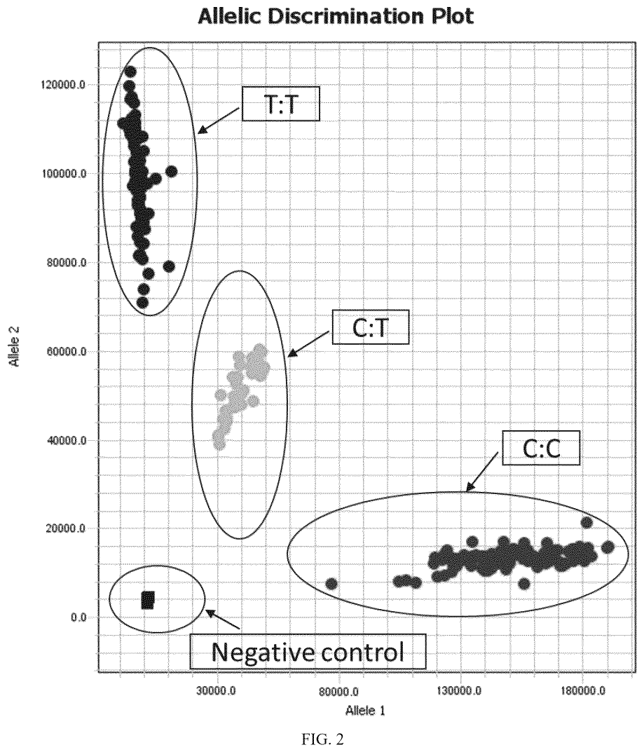

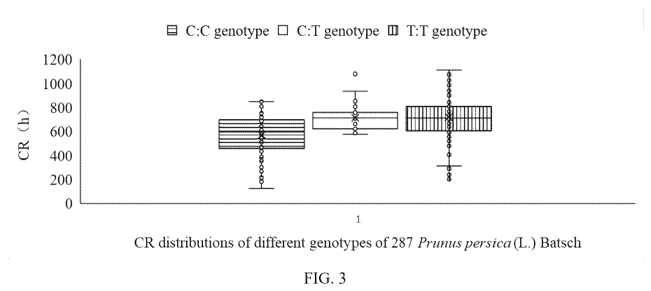

A total of three genotypes, namely, C:C, C:T, and T:T, could be detected by the KASP Chr06:26042043 primers, and relevant genotyping results were shown in FIG. 2 to FIG. 3 and Table 2. It can be seen from FIG. 2 that a genotype is determined by detecting two fluorescence intensities in a KASP product. In this figure, each dot represents a material to be tested, and a genotype represented by a class of dots is marked for ease of observation. In this figure, dots represent genotypes C:C, T:T, or C:T, and squares represent the negative control. Among the 287 samples, 149 have a C:C genotype, 96 have a T:T genotype, and 42 have a C:T genotype.

TABLE 2
CRs of Prunus persica (L.) Batsch to be tested
and corresponding genotypes determined by the
KASP molecular marker Chr06:26042043

| No. | Variety name | CR (h) | Genotype |
|---|---|---|---|
| 1 | Armking | 734 | C:C |
| 2 | BYdop7029 | 741 | C:C |
| 3 | Crimsonbaby | 301 | C:C |
| 4 | CrimsonGold | 544 | C:C |
| 5 | Flordacrest | 238 | C:C |
| 6 | Flordaglo | 232 | C:C |
| 7 | Flordaking | 238 | T:T |
| 8 | Sunraycer | 238 | C:C |
| 9 | Sunsblaze | 287 | C:C |
| 10 | Sunsplash | 317 | C:C |
| 11 | TX2B7N | 287 | C:C |
| 12 | TX2C104N | 270 | C:C |
| 13 | TX4C188LWN | 313 | T:T |
| 14 | TX4C189LN | 598 | C:C |
| 15 | TX4F244C | 330 | C:C |
| 16 | Ali Mountain Maotao | 233 | C:C |
| 17 | Amusiting | 807 | T:T |
| 18 | Aiweici | 815 | C:C |
| 19 | Annong Shuimi | 708 | C:T |
| 20 | Ba 6 | 272 | C:C |
| 21 | Baihua Bitao | 405 | T:T |
| 22 | Baihua Shanbitao | 405 | T:T |
| 23 | Baihua Shuimi | 730 | C:C |
| 24 | Baimang Pantao | 586 | C:C |
| 25 | Baimi Pantao | 696 | C:C |
| 26 | Baixianglu | 754 | C:T |
| 27 | Banjin Tao | 586 | C:T |
| 28 | Bopi Yangtao | 754 | T:T |
| 29 | Beijing Yixianhong | 912 | T:T |
| 30 | Beinong No. 1 | 712 | T:T |
| 31 | Beinong No. 2 | 754 | T:T |
| 32 | Bixia Pantao | 724 | C:C |
| 33 | Bumuzaosheng | 712 | C:T |
| 34 | Cangfangzaosheng | 777 | C:T |
| 35 | Chao Wuyuehuo | 322 | C:C |
| 36 | Zhaoxia | 741 | T:T |
| 37 | Chenpu Pantao | 696 | C:C |
| 38 | Chiyuanmi | 772 | T:T |
| 39 | Chuxiangmei | 807 | C:T |
| 40 | Chunlei | 714 | C:T |
| 41 | Chunmi | 276 | C:C |
| 42 | Chunshi | 435 | T:T |
| 43 | Chunxiami | 754 | C:T |
| 44 | Dahezaosheng | 807 | T:T |
| 45 | Dahongpao | 613 | T:T |
| 46 | Dahongpao-xin | 659 | C:C |
| 47 | Dahujing | 611 | C:T |
| 48 | Dajindan | 294 | C:C |
| 49 | Dajiubao | 732 | C:T |
| 50 | Dashutao | 357 | C:C |
| 51 | Datiantao | 749 | C:C |
| 52 | Datuan Milu | 570 | C:C |
| 53 | Daye Huanggantao | 712 | T:T |
| 54 | Danbanbaihua | 554 | T:T |
| 55 | German Huangjin | 696 | C:C |
| 56 | Dikesong | 696 | C:C |
| 57 | Dianhuabaifeng | 581 | C:C |
| 58 | Diaozhibai | 752 | C:C |
| 59 | Erzaotao | 556 | C:C |
| 60 | Prunus persica ‘Magnifica’ | 616 | T:T |
| 61 | Feicheng 1-72 | 990 | T:T |
| 62 | Feicheng No. 17 | 983 | T:T |
| 63 | Feicheng No. 39 | 1078 | C:T |
| 64 | Feicheng No. 47 | 913 | T:T |
| 65 | Feicheng 51-28 | 1112 | T:T |
| 66 | Feicheng 5182 | 1037 | T:T |
| 67 | Feicheng 5311 | 1024 | T:T |
| 68 | Feicheng Baili | 1037 | T:T |
| 69 | Fenghuang | 573 | C:T |
| 70 | Fenghua Pantao | 750 | C:C |
| 71 | Fenghuayulu (late) | 751 | T:T |
| 72 | Fenghuayulu (early) | 544 | T:T |
| 73 | Frederick | 495 | C:C |
| 74 | Fortuna | 696 | C:C |
| 75 | Prunus kansuensis Rehder No. 2 | 289 | T:T |
| 76 | Okayama zaosheng | 675 | T:T |
| 77 | Gaopobi Maotao No. 1 | 183 | C:C |
| 78 | Guantao No. 14 | 696 | C:C |
| 79 | Guantao No. 5 | 629 | C:C |
| 80 | Guizhou Qingtao | 728 | C:T |
| 81 | Harvard | 696 | C:C |
| 82 | Haluhong | 835 | C:C |
| 83 | Hanlumi | 812 | C:T |
| 84 | Black Prunus persica var. | 608 | C:C |
| | nectarina Yeseshen | | |
| 85 | Hongchuizhi | 603 | T:T |
| 86 | Hongguojian | 675 | C:T |
| 87 | Hongroutao No. 1 | 633 | C:C |
| 88 | Hongye winter peach | 494 | T:T |
| 89 | Hongye Tao | 554 | T:T |
| 90 | Hujingmilu | 667 | T:T |
| 91 | Hu 021 | 724 | T:T |
| 92 | Huayulu | 900 | T:T |
| 93 | Huanggan No. 4 | 754 | C:C |
| 94 | Huanggan No. 5 | 754 | C:C |
| 95 | Huangjinmeili | 601 | T:T |
| 96 | Huanglu Pantao | 754 | C:C |
| 97 | Huangmitao | 385 | C:C |
| 98 | Huangxintao | 385 | C:C |
| 99 | Huangyan | 696 | C:C |
| 100 | Huangzhanhe | 509 | T:T |
| 101 | Huangzhanhu | 385 | C:C |
| 102 | Hunchuntao No. 1 | 790 | T:T |
| 103 | Hunchuntao No. 2 | 807 | T:T |
| 104 | Hunchuntao No. 3 | 705 | T:T |
| 105 | Huiyulu | 650 | C:C |
| 106 | Huoxiang Huangjin Tao | 696 | C:C |
| 107 | Huozhu | 720 | T:T |
| 108 | Jinadikesi No. 4 | 807 | C:C |
| 109 | Jinadikesi No. 7 | 754 | C:T |
| 110 | Jiaqing Pantao | 696 | C:C |
| 111 | Jiangjin IV2-9 | 754 | C:T |
| 112 | Jiangjin IV3-9 | 754 | T:T |
| 113 | Jiang Tao | 667 | T:T |
| 114 | Jie Tao | 700 | T:T |
| 115 | Jinfeng | 556 | C:C |
| 116 | Jinhualu | 669 | C:T |
| 117 | Golden Queen | 828 | C:C |
| 118 | Jinhui | 603 | C:T |
| 119 | Jinqian Pantao | 769 | C:C |
| 120 | Jinshan Zaohong | 659 | C:C |
| 121 | Jinshan Zaolu | 597 | C:T |
| 122 | Jintong No. 5 | 732 | C:C |
| 123 | Jintong No. 7 | 754 | C:C |
| 124 | Jintong No. 8 | 754 | C:C |
| 125 | Jintong No. 9 | 712 | C:C |
| 126 | Jinxia Pantao | 585 | C:C |
| 127 | Jinxu | 686 | C:C |
| 128 | Jinying | 321 | C:C |
| 129 | Jinluoman | 754 | C:C |
| 130 | Jinxiang | 663 | C:T |
| 131 | Jinxiu | 705 | T:T |
| 132 | Juzaosheng | 807 | C:C |
| 133 | Juhuatao | 1090 | T:T |
| 134 | Kashiliguang | 597 | C:C |
| 135 | Kadinuo | 712 | T:T |
| 136 | Lishuitao No. 13 | 464 | C:C |
| 137 | Lianhuang | 500 | C:C |
| 138 | Lianyungang winter peach | 849 | C:C |
| 139 | Liangyuan | 754 | C:T |
| 140 | Liuyang Sijintao | 732 | T:T |
| 141 | Liuye Feitao | 911 | T:T |
| 142 | Liuyuetuan | 591 | T:T |
| 143 | Long 1-2-4 | 712 | T:T |
| 144 | Lushui-6 | 913 | T:T |
| 145 | Lubin | 480 | T:T |
| 146 | Lulin | 696 | C:C |
| 147 | Lvhua No. 3 | 754 | T:T |
| 148 | Maliweila | 266 | C:C |
| 149 | Maixiang | 865 | T:T |
| 150 | Manchengxuetao | 947 | T:T |
| 151 | Mangxialu | 754 | C:T |
| 152 | Mangzhonglu | 728 | C:C |
| 153 | Maozitao 318 | 495 | C:C |
| 154 | Meishuo | 712 | C:T |
| 155 | Nanjing Baisha | 629 | C:C |
| 156 | Nanshan Tiantao | 201 | C:C |
| 157 | Nanshan Tiantao No. 1 | 227 | C:C |
| 158 | Nanshan Tiantao No. 2 | 216 | C:C |
| 159 | Nanshan Tiantao No. 3 | 216 | C:C |
| 160 | Nongshen | 696 | C:C |
| 161 | Pantao King | 435 | C:C |
| 162 | Pei County winter peach | 518 | T:T |
| 163 | Pingbeizi | 704 | T:T |
| 164 | Qidong Prunus persica var. | 634 | C:C |
| | nectarina | | |
| 165 | Qingye winter peach | 521 | T:T |
| 166 | Qiubaitao | 271 | C:C |
| 167 | Qiubanjin | 707 | C:C |
| 168 | Qiukui | 696 | C:C |
| 169 | Qujing Tiantao | 506 | T:T |
| 170 | Japan 86 | 754 | C:C |
| 171 | Japan 89 | 603 | C:T |
| 172 | Japan baifeng | 850 | C:T |
| 173 | Richuan baifeng | 621 | C:T |
| 174 | Roupantao | 706 | C:C |
| 175 | Ruiguang 18 | 641 | C:C |
| 176 | Ruiguangmeiyu | 593 | C:C |
| 177 | Sahuahong Pantao | 542 | C:C |
| 178 | Samenlaite | 769 | C:T |
| 179 | Shazizaosheng | 608 | C:T |
| 180 | Prunus davidiana var. potaninii | 455 | C:C |
| | (Batalin) Rehder | | |
| 181 | Shanxi Taobadan | 378 | C:C |
| 182 | Shenzhou Baimi | 924 | T:T |
| 183 | Shenzhou Hongmi | 772 | T:T |
| 184 | Shennonghongrou | 578 | T:T |
| 185 | Shuguang | 599 | C:C |
| 186 | Shuangbai | 1073 | T:T |
| 187 | Shuangxihong | 575 | C:C |
| 188 | Shuanghongmi | 919 | T:T |
| 189 | Siyueban | 719 | C:C |
| 190 | Suhong | 700 | C:C |
| 191 | Suanrou Dahongpao | 632 | C:C |
| 192 | Tasikang | 696 | C:C |
| 193 | Thai Maotao | 263 | T:T |
| 194 | Tanchun | 202 | T:T |
| 195 | Tangshan Prunus davidiana | 867 | T:T |
| 196 | East Tianjin Shuimi | 695 | C:C |
| 197 | West Tianjin Shuimi | 695 | T:T |
| 198 | Tiejin No. 1 | 684 | T:T |
| 199 | Touxinhong | 628 | C:C |
| 200 | Tubade | 553 | C:C |
| 201 | Wanbufeng | 932 | C:T |
| 202 | Wanhujin | 635 | T:T |
| 203 | Wanliangyuan | 733 | C:T |
| 204 | Wan Pantao | 733 | C:C |
| 205 | Late-maturing Dao Pantao | 740 | C:C |
| 206 | Wanshuomi | 844 | T:T |
| 207 | Weifan | 696 | C:C |
| 208 | Wuhei Jirou Tao | 307 | C:C |
| 209 | Wuyue Xian | 944 | T:T |
| 210 | Ximushi | 696 | C:C |
| 211 | Ximi | 609 | C:T |
| 212 | Xiaguang | 583 | C:C |
| 213 | Xiahui No. 1 | 585 | C:T |
| 214 | Xiahui No. 2 | 754 | C:C |
| 215 | Xiahui No. 3 | 712 | T:T |
| 216 | Xiahui No. 4 | 754 | C:C |
| 217 | Xiahui No. 5 | 546 | C:C |
| 218 | Xiahui No. 6 | 756 | C:C |
| 219 | Xiahui No. 8 | 545 | C:C |
| 220 | Xiaji Tao | 519 | C:C |
| 221 | Xiazhi Tao | 374 | C:C |
| 222 | Xiangjinpan | 712 | T:T |
| 223 | Xiangtao | 1082 | T:T |
| 224 | Xiangyabai | 756 | T:T |
| 225 | Xiaohongpao | 676 | T:T |
| 226 | Xiaojindan | 385 | C:C |
| 227 | Xiaogan LZ | 732 | T:T |
| 228 | Xinbaihua | 603 | T:T |
| 229 | Xinjiang No. 1 | 735 | C:T |
| 230 | Xinjiang No. 3 | 648 | T:T |
| 231 | Xinjiang No. 4 | 768 | T:T |
| 232 | Xinjiang No. 5 | 809 | T:T |
| 233 | Xinjiang Datianren | 601 | T:T |
| 234 | Xinjiang Huangrou | 940 | T:T |
| 235 | Xinjiang Pantao | 556 | C:C |
| 236 | Xuancheng Tiantao | 684 | C:C |
| 237 | Xuancheng Tiantao No. 2 | 635 | C:T |
| 238 | Xuebaitao | 556 | C:C |
| 239 | Yanzhitao | 541 | C:C |
| 240 | Yangtao No. 40 | 704 | C:T |
| 241 | Yangzhou early-sweet peach | 646 | T:T |
| 242 | Yejihong | 699 | T:T |
| 243 | Yixianhong | 695 | C:C |
| 244 | Yinhe | 385 | C:C |
| 245 | Yinhualu | 657 | T:T |
| 246 | Indian Hongrou | 539 | C:C |
| 247 | Yingqing | 769 | C:T |
| 248 | You Pantao | 754 | C:C |
| 249 | Yuyi | 807 | T:T |
| 250 | Yuhualu | 805 | C:T |
| 251 | Yulu Pantao | 548 | C:C |
| 252 | Yuxia Pantao | 470 | C:C |
| 253 | Yunlong Shuimi | 807 | T:T |
| 254 | Yunnan Tao No. 41 | 591 | C:C |
| 255 | Yunnan Tao No. 42 | 741 | T:T |
| 256 | Yunnan Tao No. 43 | 696 | C:C |
| 257 | Yunnan Tao No. 44 | 807 | T:T |
| 258 | Yunnan Tao No. 45 | 849 | C:C |
| 259 | Yunnan Tao No. 47 | 696 | C:C |
| 260 | Yunnan Tao No. 49 | 547 | C:C |
| 261 | Yunnan Tao No. 51 | 509 | C:C |
| 262 | Yuntai Mountain Prunus davidiana | 391 | C:C |
| 263 | Zaobaihua | 662 | C:T |
| 264 | Zaofengwang | 638 | C:T |
| 265 | Zaohualu | 707 | T:T |
| 266 | Zaohuang Pantao | 353 | C:C |
| 267 | Zaojinlu | 435 | C:C |
| 268 | Zaokuimi | 601 | C:C |
| 269 | Zaolu Pantao | 586 | C:C |
| 270 | Zaomei | 709 | T:T |
| 271 | Zaoshuomi | 524 | C:C |
| 272 | Changsheng Pantao | 696 | C:C |
| 273 | Zhengji | 611 | C:T |
| 274 | Zhonghujing | 581 | T:T |
| 275 | Zhonghua shoutao | 970 | T:T |
| 276 | Zhongshan | 712 | C:T |
| 277 | Broom-shaped Prunus davidiana | 126 | C:C |
| 278 | Tsukuba No. 5 | 654 | T:T |
| 279 | Zijinhong No. 2 | 588 | C:C |
| 280 | Purple Maotao 480 | 574 | C:C |
| 281 | Purple Prunus persica var. | 591 | C:C |
| | nectarina No. 1 | | |
| 282 | Purple Prunus persica var. | 564 | C:C |
| | nectarina No. 3 | | |
| 283 | Purple Prunus persica var. | 552 | C:C |
| | nectarina No. 4 | | |
| 284 | Purple Prunus persica var. | 646 | C:C |
| | nectarina No. 5 | | |
| 285 | Purple Prunus persica var. | 633 | C:C |
| | nectarina No. 6 | | |
| 286 | Purple Prunus persica var. | 556 | C:C |
| | nectarina No. 7 | | |
| 287 | Purple Prunus persica var. | 556 | C:C |
| | nectarina No. 9 | | |

It can be seen from FIG. 3 and Table 2 that an average CR of Prunus persica (L.) Batsch with a genotype C:C is 569 h, an average CR of Prunus persica (L.) Batsch with a genotype C:T is 711 h, and an average CR of Prunus persica (L.) Batsch with a genotype T:T is 719 h. According to independent-sample t-test results, CRs of Prunus persica (L.) Batsch with genotypes C:T and T:T are not significantly different from each other, but both are extremely significantly higher than a CR of Prunus persica (L.) Batsch with a genotype C:C (p<0.01), with a statistical significance. There are 149 Prunus persica (L.) Batsch materials with a genotype C:C, including 34 Prunus persica (L.) Batsch materials with a low CR, 43 Prunus persica (L.) Batsch materials with a medium CR, 72 Prunus persica (L.) Batsch materials with a high CR, and 0 Prunus persica (L.) Batsch material with an extremely high CR. There are 139 Prunus persica (L.) Batsch materials with genotypes C:T and T:T, including 5 Prunus persica (L.) Batsch materials with a low CR, 19 Prunus persica (L.) Batsch materials with a medium CR, 93 Prunus persica (L.) Batsch materials with a high CR, and 22 Prunus persica (L.) Batsch materials with an extremely high CR.

### Example 3 Use of the KASP Molecular Marker Chr01:46470090

A total of 287 Prunus persica (L.) Batsch materials with rich genetic contexts (including cultivated varieties, local varieties, and wild resources from China, the United States, Thailand, etc., and belonging to germplasm types such as Prunus persica var. persica, Prunus kansuensis Rehder, Prunus davidiana, and Prunus ferganensis) were adopted as test materials. CRs of these test materials were determined (Table 3). 39 of the test materials had a low CR, 62 of the test materials had a medium CR, 164 of the test materials had a high CR, and 22 of the test materials had an extremely high CR. DNA was extracted from the 287 Prunus persica (L.) Batsch materials by a CTAB method, and the entire batch of DNA samples was diluted proportionally to a concentration of 1 ng/μL to 9 ng/μL. The KASP Chr01:46470090 primers provided in Example 1 were used for the genotyping of Prunus persica (L.) Batsch. A reaction system was as follows:
a 5 μL PCR system including 2.5 μL of 2*KASP master mix, 1.25 μL of the primer combination, and 1.25 μL of a diluted sample DNA (notes: in a negative control, the sample DNA was replaced with 1.25 μL of the buffer TE; and in a positive control, a Prunus persica (L.) Batsch material with a known loci SNP genotyping result produced based on first-generation sequencing (reverse sequencing) was adopted (which was included in the above 287 Prunus persica (L.) Batsch materials, and included “Flordaglo” with a C:C genotype and “Feicheng Baili” with a T:T genotype). PCR analysis was conducted according to the instructions of the CFX Connect™ Real-Time System (Bio-Rad, USA) instrument. A PCR procedure was as follows: pre-denaturation at 95° C. for 10 min, 1 cycle; denaturation at 95° C. for 20 s; annealing-extension at 61° C. to 55° C. for 60 s, 10 cycles; denaturation at 95° C. for 20 s, and annealing-extension at 55° C. for 60 s, 27 cycles; and reading at 25° C. for 30 s, 1 cycle.

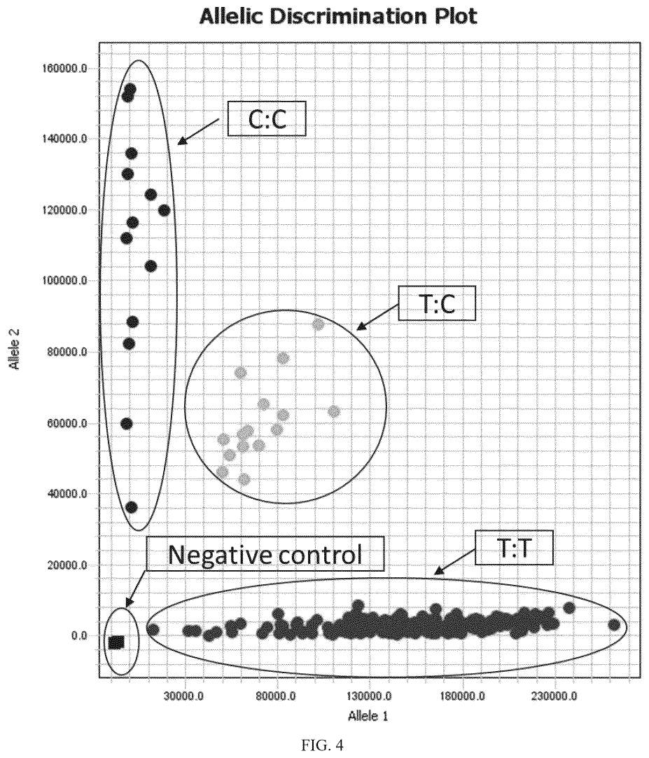

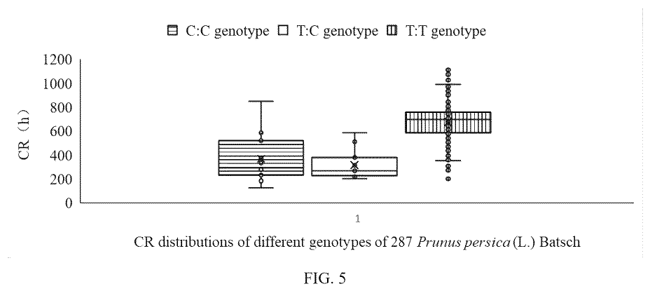

A total of three genotypes, namely, T:T, T:C, and C:C, could be detected by the KASP Chr01:46470090 primers, and relevant results were shown in FIG. 4 to FIG. 5 and Table 3. It can be seen from FIG. 4 that a genotype is determined by detecting two fluorescence intensities in a KASP product. In this figure, each dot represents a material to be tested, and a genotype represented by a class of dots is marked for ease of observation. In this figure, dots represent genotypes T:T, C:C, or T:C, and squares represent the negative control. 259 have a T:T genotype, 13 have a C:C genotype, and 15 have a T:C genotype.

TABLE 3
CRs of Prunus persica (L.) Batsch to be tested
and corresponding genotypes determined by the
KASP molecular marker Chr01:46470090

| No. | Variety name | CR (h) | Genotype |
|---|---|---|---|
| 1 | Armking | 734 | T:T |
| 2 | BYdop7029 | 741 | T:T |
| 3 | Crimsonbaby | 301 | T:T |
| 4 | CrimsonGold | 544 | T:T |
| 5 | Flordacrest | 238 | T:C |
| 6 | Flordaglo | 232 | C:C |
| 7 | Flordaking | 238 | T:C |
| 8 | Sunraycer | 238 | T:C |
| 9 | Sunsblaze | 287 | T:C |
| 10 | Sunsplash | 317 | T:T |
| 11 | TX2B7N | 287 | T:T |
| 12 | TX2C104N | 270 | T:T |
| 13 | TX4C188LWN | 313 | T:C |
| 14 | TX4C189LN | 598 | T:T |
| 15 | TX4F244C | 330 | C:C |
| 16 | Ali Mountain Maotao | 233 | C:C |
| 17 | Amusiting | 807 | T:T |
| 18 | Aiweici | 815 | T:T |
| 19 | Annong Shuimi | 708 | T:T |
| 20 | Ba 6 | 272 | T:C |
| 21 | Baihua Bitao | 405 | T:T |
| 22 | Baihua Shanbitao | 405 | T:T |
| 23 | Baihua Shuimi | 730 | T:T |
| 24 | Baimang Pantao | 586 | T:T |
| 25 | Baimi Pantao | 696 | T:T |
| 26 | Baixianglu | 754 | T:T |
| 27 | Banjin Tao | 586 | T:C |
| 28 | Bopi Yangtao | 754 | T:T |
| 29 | Beijing Yixianhong | 912 | T:T |
| 30 | Beinong No. 1 | 712 | T:T |
| 31 | Beinong No. 2 | 754 | T:T |
| 32 | Bixia Pantao | 724 | T:T |
| 33 | Bumuzaosheng | 712 | T:T |
| 34 | Cangfangzaosheng | 777 | T:T |
| 35 | Chao Wuyuehuo | 322 | T:T |
| 36 | Zhaoxia | 741 | T:T |
| 37 | Chenpu Pantao | 696 | T:T |
| 38 | Chiyuanmi | 772 | T:T |
| 39 | Chuxiangmei | 807 | T:T |
| 40 | Chunlei | 714 | T:T |
| 41 | Chunmi | 276 | C:C |
| 42 | Chunshi | 435 | T:T |
| 43 | Chunxiami | 754 | T:T |
| 44 | Dahezaosheng | 807 | T:T |
| 45 | Dahongpao | 613 | T:T |
| 46 | Dahongpao-xin | 659 | T:T |
| 47 | Dahujing | 611 | T:T |
| 48 | Dajindan | 294 | T:T |
| 49 | Dajiubao | 732 | T:T |
| 50 | Dashutao | 357 | T:T |
| 51 | Datiantao | 749 | T:T |
| 52 | Datuan Milu | 570 | T:T |
| 53 | Daye Huanggantao | 712 | T:T |
| 54 | Danbanbaihua | 554 | T:T |
| 55 | German Huangjin | 696 | T:T |
| 56 | Dikesong | 696 | T:T |
| 57 | Dianhuabaifeng | 581 | T:T |
| 58 | Diaozhibai | 752 | T:T |
| 59 | Erzaotao | 556 | T:T |
| 60 | Prunus persica ‘Magnifica’ | 616 | T:T |
| 61 | Feicheng 1-72 | 990 | T:T |
| 62 | Feicheng No. 17 | 983 | T:T |
| 63 | Feicheng No. 39 | 1078 | T:T |
| 64 | Feicheng No. 47 | 913 | T:T |
| 65 | Feicheng 51-28 | 1112 | T:T |
| 66 | Feicheng 5182 | 1037 | T:T |
| 67 | Feicheng 5311 | 1024 | T:T |
| 68 | Feicheng Baili | 1037 | T:T |
| 69 | Fenghuang | 573 | T:T |
| 70 | Fenghua Pantao | 750 | T:T |
| 71 | Fenghuayulu (late) | 751 | T:T |
| 72 | Fenghuayulu (early) | 544 | T:T |
| 73 | Frederick | 495 | T:T |
| 74 | Fortuna | 696 | T:T |
| 75 | Prunus kansuensis Rehder No. 2 | 289 | C:C |
| 76 | Okayama zaosheng | 675 | T:T |
| 77 | Gaopobi Maotao No. 1 | 183 | C:C |
| 78 | Guantao No. 14 | 696 | T:T |
| 79 | Guantao No. 5 | 629 | T:T |
| 80 | Guizhou Qingtao | 728 | T:T |
| 81 | Harvard | 696 | T:T |
| 82 | Haluhong | 835 | T:T |
| 83 | Hanlumi | 812 | T:T |
| 84 | Black Prunus persica var. | 608 | T:T |
| | nectarina Yeseshen | | |
| 85 | Hongchuizhi | 603 | T:T |
| 86 | Hongguojian | 675 | T:T |
| 87 | Hongroutao No. 1 | 633 | T:T |
| 88 | Hongye winter peach | 585 | C:C |
| 89 | Hongye Tao | 554 | T:T |
| 90 | Hujingmilu | 667 | T:T |
| 91 | Hu 021 | 724 | T:T |
| 92 | Huayulu | 900 | T:T |
| 93 | Huanggan No. 4 | 754 | T:T |
| 94 | Huanggan No. 5 | 754 | T:T |
| 95 | Huangjinmeili | 601 | T:T |
| 96 | Huanglu Pantao | 754 | T:T |
| 97 | Huangmitao | 385 | T:T |
| 98 | Huangxintao | 385 | T:T |
| 99 | Huangyan | 696 | T:T |
| 100 | Huangzhanhe | 509 | T:T |
| 101 | Huangzhanhu | 385 | T:T |
| 102 | Hunchuntao No. 1 | 790 | T:T |
| 103 | Hunchuntao No. 2 | 807 | T:T |
| 104 | Hunchuntao No. 3 | 705 | T:T |
| 105 | Huiyulu | 650 | T:T |
| 106 | Huoxiang Huangjin Tao | 696 | T:T |
| 107 | Huozhu | 720 | T:T |
| 108 | Jinadikesi No. 4 | 807 | T:T |
| 109 | Jinadikesi No. 7 | 754 | T:T |
| 110 | Jiaqing Pantao | 696 | T:T |
| 111 | Jiangjin IV2-9 | 754 | T:T |
| 112 | Jiangjin IV3-9 | 754 | T:T |
| 113 | Jiang Tao | 667 | T:T |
| 114 | Jie Tao | 700 | T:T |
| 115 | Jinfeng | 556 | T:T |
| 116 | Jinhualu | 669 | T:T |
| 117 | Golden Queen | 828 | T:T |
| 118 | Jinhui | 603 | T:T |
| 119 | Jinqian Pantao | 769 | T:T |
| 120 | Jinshan Zaohong | 659 | T:T |
| 121 | Jinshan Zaolu | 597 | T:T |
| 122 | Jintong No. 5 | 732 | T:T |
| 123 | Jintong No. 7 | 754 | T:T |
| 124 | Jintong No. 8 | 754 | T:T |
| 125 | Jintong No. 9 | 712 | T:T |
| 126 | Jinxia Pantao | 585 | T:T |
| 127 | Jinxu | 686 | T:T |
| 128 | Jinying | 321 | T:T |
| 129 | Jinluoman | 754 | T:T |
| 130 | Jinxiang | 663 | T:T |
| 131 | Jinxiu | 705 | T:T |
| 132 | Juzaosheng | 807 | T:T |
| 133 | Juhuatao | 1090 | T:T |
| 134 | Kashiliguang | 597 | T:T |
| 135 | Kadinuo | 712 | T:T |
| 136 | Lishuitao No. 13 | 464 | T:T |
| 137 | Lianhuang | 500 | T:T |
| 138 | Lianyungang winter peach | 849 | T:T |
| 139 | Liangyuan | 754 | T:T |
| 140 | Liuyang Sijintao | 732 | T:T |
| 141 | Liuye Feitao | 911 | T:T |
| 142 | Liuyuetuan | 591 | T:T |
| 143 | Long 1-2-4 | 712 | T:T |
| 144 | Lushui-6 | 913 | T:T |
| 145 | Lubin | 480 | T:T |
| 146 | Lulin | 696 | T:T |
| 147 | Lvhua No. 3 | 754 | T:T |
| 148 | Maliweila | 266 | T:C |
| 149 | Maixiang | 865 | T:T |
| 150 | Manchengxuetao | 947 | T:T |
| 151 | Mangxialu | 754 | T:T |
| 152 | Mangzhonglu | 728 | T:T |
| 153 | Maozitao 318 | 495 | T:T |
| 154 | Meishuo | 712 | T:T |
| 155 | Nanjing Baisha | 629 | T:T |
| 156 | Nanshan Tiantao | 201 | T:C |
| 157 | Nanshan Tiantao No. 1 | 227 | T:C |
| 158 | Nanshan Tiantao No. 2 | 216 | T:C |
| 159 | Nanshan Tiantao No. 3 | 216 | T:C |
| 160 | Nongshen | 696 | T:T |
| 161 | Pantao King | 435 | T:T |
| 162 | Pei County winter peach | 518 | C:C |
| 163 | Pingbeizi | 704 | T:T |
| 164 | Qidong Prunus persica var. | 634 | T:T |
| | nectarina | | |
| 165 | Qingye winter peach | 521 | T:C |
| 166 | Qiubaitao | 271 | T:T |
| 167 | Qiubanjin | 707 | T:T |
| 168 | Qiukui | 696 | T:T |
| 169 | Qujing Tiantao | 506 | T:T |
| 170 | Japan 86 | 754 | T:T |
| 171 | Japan 89 | 603 | T:T |
| 172 | Japan baifeng | 850 | T:T |
| 173 | Richuan baifeng | 621 | T:T |
| 174 | Roupantao | 706 | T:T |
| 175 | Ruiguang 18 | 641 | T:T |
| 176 | Ruiguangmeiyu | 593 | T:T |
| 177 | Sahuahong Pantao | 542 | T:T |
| 178 | Samenlaite | 769 | T:T |
| 179 | Shazizaosheng | 608 | T:T |
| 180 | Prunus davidiana var. potaninii | 455 | T:T |
| | (Batalin) Rehder | | |
| 181 | Shanxi Taobadan | 378 | T:C |
| 182 | Shenzhou Baimi | 924 | T:T |
| 183 | Shenzhou Hongmi | 772 | T:T |
| 184 | Shennonghongrou | 578 | T:T |
| 185 | Shuguang | 599 | T:T |
| 186 | Shuangbai | 1073 | T:T |
| 187 | Shuangxihong | 575 | T:T |
| 188 | Shuanghongmi | 919 | T:T |
| 189 | Siyueban | 719 | T:T |
| 190 | Suhong | 700 | T:T |
| 191 | Suanrou Dahongpao | 632 | T:T |
| 192 | Tasikang | 696 | T:T |
| 193 | Thai Maotao | 263 | C:C |
| 194 | Tanchun | 202 | T:T |
| 195 | Tangshan Prunus davidiana | 867 | T:T |
| 196 | East Tianjin Shuimi | 695 | T:T |
| 197 | West Tianjin Shuimi | 695 | T:T |
| 198 | Tiejin No. 1 | 684 | T:T |
| 199 | Touxinhong | 628 | T:T |
| 200 | Tubade | 553 | T:T |
| 201 | Wanbufeng | 932 | T:T |
| 202 | Wanhujin | 635 | T:T |
| 203 | Wanliangyuan | 733 | T:T |
| 204 | Wan Pantao | 733 | T:T |
| 205 | Late-maturing Dao Pantao | 740 | T:T |
| 206 | Wanshuomi | 844 | T:T |
| 207 | Weifan | 696 | T:T |
| 208 | Wuhei Jirou Tao | 307 | T:T |
| 209 | Wuyue Xian | 944 | T:T |
| 210 | Ximushi | 696 | T:T |
| 211 | Ximi | 609 | T:T |
| 212 | Xiaguang | 583 | T:T |
| 213 | Xiahui No. 1 | 585 | T:T |
| 214 | Xiahui No. 2 | 754 | T:T |
| 215 | Xiahui No. 3 | 712 | T:T |
| 216 | Xiahui No. 4 | 754 | T:T |
| 217 | Xiahui No. 5 | 546 | T:T |
| 218 | Xiahui No. 6 | 756 | T:T |
| 219 | Xiahui No. 8 | 545 | T:T |
| 220 | Xiaji Tao | 519 | C:C |
| 221 | Xiazhi Tao | 374 | C:C |
| 222 | Xiangjinpan | 712 | T:T |
| 223 | Xiangtao | 1082 | T:T |
| 224 | Xiangyabai | 756 | T:T |
| 225 | Xiaohongpao | 676 | T:T |
| 226 | Xiaojindan | 385 | T:T |
| 227 | Xiaogan LZ | 732 | T:T |
| 228 | Xinbaihua | 603 | T:T |
| 229 | Xinjiang No. 1 | 735 | T:T |
| 230 | Xinjiang No. 3 | 648 | T:T |
| 231 | Xinjiang No. 4 | 768 | T:T |
| 232 | Xinjiang No. 5 | 809 | T:T |
| 233 | Xinjiang Datianren | 601 | T:T |
| 234 | Xinjiang Huangrou | 940 | T:T |
| 235 | Xinjiang Pantao | 556 | T:T |
| 236 | Xuancheng Tiantao | 684 | T:T |
| 237 | Xuancheng Tiantao No. 2 | 635 | T:T |
| 238 | Xuebaitao | 556 | T:T |
| 239 | Yanzhitao | 541 | T:T |
| 240 | Yangtao No. 40 | 704 | T:T |
| 241 | Yangzhou early-sweet peach | 646 | T:T |
| 242 | Yejihong | 699 | T:T |
| 243 | Yixianhong | 695 | T:T |
| 244 | Yinhe | 385 | T:T |
| 245 | Yinhualu | 657 | T:T |
| 246 | Indian Hongrou | 539 | T:T |
| 247 | Yingqing | 769 | T:T |
| 248 | You Pantao | 754 | T:T |
| 249 | Yuyi | 807 | T:T |
| 250 | Yuhualu | 805 | T:T |
| 251 | Yulu Pantao | 548 | T:T |
| 252 | Yuxia Pantao | 470 | T:T |
| 253 | Yunlong Shuimi | 807 | T:T |
| 254 | Yunnan Tao No. 41 | 591 | T:T |
| 255 | Yunnan Tao No. 42 | 741 | T:T |
| 256 | Yunnan Tao No. 43 | 696 | T:T |
| 257 | Yunnan Tao No. 44 | 807 | T:T |
| 258 | Yunnan Tao No. 45 | 849 | C:C |
| 259 | Yunnan Tao No. 47 | 696 | T:T |
| 260 | Yunnan Tao No. 49 | 547 | T:T |
| 261 | Yunnan Tao No. 51 | 509 | T:C |
| 262 | Yuntai Mountain Prunus davidiana | 391 | T:T |
| 263 | Zaobaihua | 662 | T:T |
| 264 | Zaofengwang | 638 | T:T |
| 265 | Zaohualu | 707 | T:T |
| 266 | Zaohuang Pantao | 353 | T:T |
| 267 | Zaojinlu | 435 | T:T |
| 268 | Zaokuimi | 601 | T:T |
| 269 | Zaolu Pantao | 586 | T:T |
| 270 | Zaomei | 709 | T:T |
| 271 | Zaoshuomi | 524 | T:T |
| 272 | Changsheng Pantao | 696 | T:T |
| 273 | Zhengji | 611 | T:T |
| 274 | Zhonghujing | 581 | T:T |
| 275 | Zhonghua shoutao | 970 | T:T |
| 276 | Zhongshan Zaolu | 712 | T:T |
| 277 | Broom-shaped Prunus davidiana | 126 | C:C |
| 278 | Tsukuba No. 5 | 654 | T:T |
| 279 | Zijinhong No. 2 | 588 | T:T |
| 280 | Purple Maotao 480 | 574 | T:T |
| 281 | Purple Prunus persica var. | 591 | T:T |
| | nectarina No. 1 | | |
| 282 | Purple Prunus persica var. | 564 | T:T |
| | nectarina No. 3 | | |
| 283 | Purple Prunus persica var. | 552 | T:T |
| | nectarina No. 4 | | |
| 284 | Purple Prunus persica var. | 646 | T:T |
| | nectarina No. 5 | | |
| 285 | Purple Prunus persica var. | 633 | T:T |
| | nectarina No. 6 | | |
| 286 | Purple Prunus persica var. | 556 | T:T |
| | nectarina No. 7 | | |
| 287 | Purple Prunus persica var. | 556 | T:T |
| | nectarina No. 9 | | |

It can be seen from FIG. 5 and Table 3 that an average CR of Prunus persica (L.) Batsch with a genotype C:C is 367 h, an average CR of Prunus persica (L.) Batsch with a genotype T:C is 314 h, and an average CR of Prunus persica (L.) Batsch with a genotype T:T is 673 h. According to independent-sample t-test results, CRs of Prunus persica (L.) Batsch with genotypes C:C and T:C are not significantly different from each other, but both are extremely significantly lower than a CR of Prunus persica (L.) Batsch with a genotype T:T (p<0.01), with statistical significance. There are 28 Prunus persica (L.) Batsch materials with genotypes C:C and T:C, including 21 Prunus persica (L.) Batsch materials with a low CR, 6 Prunus persica (L.) Batsch materials with a medium CR, 1 Prunus persica (L.) Batsch material with a high CR, and 0 Prunus persica (L.) Batsch material with an extremely high CR. There are 259 Prunus persica (L.) Batsch materials with a genotype T:T, including 18 Prunus persica (L.) Batsch materials with a low CR, 56 Prunus persica (L.) Batsch materials with a medium CR, 163 Prunus persica (L.) Batsch materials with a high CR, and 22 Prunus persica (L.) Batsch materials with an extremely high CR.

TABLE 4
Genotyping effects of the two molecular markers for CRs of Prunus persica (L.) Batsch

| | | | | Prunus persica (L.) | | Prunus persica (L.) | | Prunus persica (L.) | |
|---|---|---|---|---|---|---|---|---|---|
| | | Prunus persica (L.) | | Batsch with a | | Batsch with a high | | Batsch with an | |
| | | Batsch with a low | | medium CR (400 | | CR (600 h ≤ CR < | | extremely high CR | |
| | | CR (CR < 400 h) | | h ≤ CR < 600 h) | | 900 h) | | (CR ≥ 900 h) | |
| | | | Identifi- | | Identifi- | | Identifi- | | Identifi- |
| KASP | | | cation | | cation | | cation | | cation |
| molecular | | Propor- | success | Propor- | success | Propor- | success | Propor- | success |
| marker | Genotype | tion (%) | rate (%) | tion (%) | rate (%) | tion (%) | rate %) | tion (%) | rate (%) |
| Chr06:26042043 | C:C | 22.8 | 87.2 | 28.9 | 69.4 | 48.3 | 43.9 | 0 | 0 |
| | C:T | 3.6 | 12.8 | 13.7 | 30.6 | 66.9 | 56.1 | 15.8 | 100 |
| | and | | | | | | | | |
| | T:T | | | | | | | | |
| Chr01:46470090 | C:C | 75.0 | 53.8 | 21.4 | 9.7 | 3.6 | 0.6 | 0 | 0 |
| | and | | | | | | | | |
| | T:C | | | | | | | | |
| | T:T | 7.0 | 46.2 | 21.6 | 90.3 | 62.9 | 99.4 | 8.5 | 100 |

Notes: A proportion refers to a proportion of a number of Prunus persica (L.) Batsch samples with a CR level that are identified by a genotype of a marker in a total number of Prunus persica (L.) Batsch samples that are identified by the genotype of the maker and is equal to a number of Prunus persica (L.) Batsch samples with a CR level that are identified by a genotype of a marker/a total number of Prunus persica (L.) Batsch samples that are identified by the genotype of the maker*100%. An identification success rate refers to a success probability that a Prunus persica (L.) Batsch material with a CR level can be identified by a genotype of a marker and is equal to a number of Prunus persica (L.) Batsch samples with a CR level that are identified by a genotype of a marker/an actual total number of Prunus persica (L.) Batsch samples with the CR level*100%.

Based on the data analysis and interpretation in Table 4, the identification effects of different genotypes of the two KASP molecular markers (Chr06:26042043 and Chr01:46470090) for Prunus persica (L.) Batsch samples with different CRs are clearly summarized.

Among C:C genotype samples marked by Chr06:26042043: A proportion of Prunus persica (L.) Batsch samples with the low CR is 22.8%, and an identification success rate of Prunus persica (L.) Batsch with the low CR is 87.2%, indicating that the C:C genotype is very suitable for the breeding of Prunus persica (L.) Batsch with the low CR. A proportion of Prunus persica (L.) Batsch samples with the medium CR is 28.9%, and an identification success rate is 69.4%, indicating that the C:C genotype also has a prominent identification ability for Prunus persica (L.) Batsch with the medium CR. A proportion of Prunus persica (L.) Batsch samples with the high CR is 48.3%, and an identification success rate is reduced to 43.9%, indicating that the C:C genotype has a relatively low identification ability for Prunus persica (L.) Batsch with the high CR. A proportion of Prunus persica (L.) Batsch samples with the extremely high CR is 0%, and thus the C:C genotype cannot identify Prunus persica (L.) Batsch with the extremely high CR. Therefore, an identification effect of the C:C genotype is mainly concentrated in Prunus persica (L.) Batsch with the low and medium CRs, and an identification ability of the C:C genotype is gradually weakened with the increase of a CR. In particular, the C:C genotype cannot identify Prunus persica (L.) Batsch with the extremely high CR.

Among C:T and T:T genotype samples marked by Chr06:26042043: A proportion of Prunus persica (L.) Batsch samples with the low CR is only 3.6%, and an identification success rate is also only 12.8%, indicating that the C:T and T:T genotypes do not have a prominent identification ability for Prunus persica (L.) Batsch with the low CR. A proportion of Prunus persica (L.) Batsch samples with the medium CR is 13.7%, and an identification success rate is 30.6%, indicating that the C:T and T:T genotypes also have a limited identification effect for Prunus persica (L.) Batsch with the medium CR. A proportion of Prunus persica (L.) Batsch samples with the high CR increases to 66.9%, and an identification success rate is 56.1%, indicating that the C:T and T:T genotypes have a prominent identification effect for Prunus persica (L.) Batsch with the high CR. Although a proportion of Prunus persica (L.) Batsch samples with the extremely high CR is only 15.8%, an identification success rate reaches 100%, indicating that the C:T and T:T genotypes are very suitable for identifying Prunus persica (L.) Batsch with the extremely high CR. Therefore, the C:T and T:T genotypes are mainly used for the identification of Prunus persica (L.) Batsch with the high CR and extremely high CR, and exhibit a very prominent identification effect for Prunus persica (L.) Batsch with the extremely high CR.

Among C:C and T:C genotype samples marked by Chr01:46470090: A proportion of Prunus persica (L.) Batsch samples with the low CR is as high as 75%, and an identification success rate is 53.8%, indicating that the C:C and T:C genotypes are suitable for the screening of Prunus persica (L.) Batsch with the low CR. A proportion of Prunus persica (L.) Batsch samples with the medium CR is 21.4%, but an identification success rate is merely 9.7%, indicating that the C:C and T:C genotypes have a weak identification effect for Prunus persica (L.) Batsch with the medium CR. Proportions of Prunus persica (L.) Batsch samples with the high and extremely high CRs and respective identification success rates are all very low, indicating that the C:C and T:C genotypes cannot effectively identify Prunus persica (L.) Batsch with the high CR or extremely high CR. Therefore, the C:C and T:C genotypes are suitable for the screening of Prunus persica (L.) Batsch with the low CR (CR<400 h) but exhibit a poor identification ability for Prunus persica (L.) Batsch with the medium, high, and extremely high CRs (CR≥400 h).

Among T:T genotype samples marked by Chr01:46470090: A proportion of Prunus persica (L.) Batsch samples with the low CR is 7%, and an identification success rate is 46.2%, indicating that the T:T genotype still has a specified identification ability. A proportion of Prunus persica (L.) Batsch samples with the medium CR is 21.6%, and an identification success rate is as high as 90.3%, indicating that the T:T genotype is very suitable for identifying Prunus persica (L.) Batsch with the medium CR. A proportion of Prunus persica (L.) Batsch samples with the high CR is 62.9%, and an identification success rate is 99.4%, indicating that the T:T genotype can very accurately identify Prunus persica (L.) Batsch with the high CR. Although a proportion of Prunus persica (L.) Batsch samples with a CR higher than or equal to 900 h is merely 8.5%, an identification success rate is 100%, indicating that the T:T genotype has a very high identification efficiency for Prunus persica (L.) Batsch with the extremely high CR. Therefore, the T:T genotype is suitable for the screening of Prunus persica (L.) Batsch with the medium, high, and extremely high CRs (CR≥400 h) and exhibits a very prominent identification ability, particularly for Prunus persica (L.) Batsch with the high CR and extremely high CR.

In summary, the C:C genotype marked by Chr06:26042043 and the C:C and T:C genotypes marked by Chr01:46470090 can be used for the identification of Prunus persica (L.) Batsch with the low CR, and the C:T and T:T genotypes marked by Chr06:26042043 and the T:T genotype marked by Chr01:46470090 provide very accurate identification effects for Prunus persica (L.) Batsch with the medium, high, and extremely high CRs. The two KASP molecular markers can be directly used for molecular breeding programs to accelerate a breeding process by screening Prunus persica (L.) Batsch breeding materials with different CR levels and new varieties.

During breeding, the two markers can also be used in combination to improve the identification success rate and the accurate distinguished cultivation of Prunus persica (L.) Batsch with a target CR. For example, when Prunus persica (L.) Batsch with the low CR is cultivated, the KASP molecular marker Chr06:26042043 and the KASP molecular marker Chr01:46470090 are used in combination. Prunus persica (L.) Batsch materials with the low CR are first locked with the C:C genotype (identification success rate: 87.2%) of the KASP molecular marker Chr06:26042043, and then most of the Prunus persica (L.) Batsch materials with the low CR (proportion: 75%) are accurately identified by the C:C and T:C genotypes of the KASP molecular marker Chr01:46470090, which greatly improves the probability and accuracy of acquiring Prunus persica (L.) Batsch with the low CR.

The molecular markers provided by the present disclosure will exhibit important reference and application prospects in different breeding projects in Prunus persica (L.) Batsch planting regions worldwide.

Although the present disclosure has been described as above, the present disclosure is not limited thereto. Those skilled in the art may make various changes and modifications without departing from the spirit and scope of the present disclosure. Therefore, the protection scope of the present disclosure shall be subject to the scope defined by the claims.

What is claimed is:

1. A Kompetitive allele-specific PCR (KASP) molecular marker Chr06:26042043 primer combination related to a chilling requirement (CR) of Prunus persica (L.) Batsch, comprising: a first forward primer, a second forward primer, and a first shared reverse primer, wherein the nucleotide sequence of the first forward primer is shown in SEQ ID NO: 1, the nucleotide sequence of the second forward primer is shown in SEQ ID NO: 2, and the nucleotide sequence of the first shared reverse primer is shown in SEQ ID NO: 3.

2. The KASP molecular marker Chr06:26042043 primer combination according to claim 1, wherein different fluorescent tags are added to 5′ terminus of the first forward primer and the second forward primer, respectively.

3. A KASP molecular marker Chr01:46470090 primer combination related to a CR of Prunus persica (L.) Batsch, comprising: a third forward primer, a fourth forward primer, and a second shared reverse primer, wherein the nucleotide sequence of the third forward primer is shown in SEQ ID NO: 4, the nucleotide sequence of the fourth forward primer is shown in SEQ ID NO: 5, and the nucleotide sequence of the second shared reverse primer is shown in SEQ ID NO: 6.

4. The KASP molecular marker Chr01:46470090 primer combination according to claim 3, wherein different fluorescent tags are added to 5′ terminus of the third forward primer and the fourth forward primer, respectively.

5. An identification method for a CR level of Prunus persica (L.) Batsch, comprising using the KASP molecular marker Chr06:26042043 primer combination according to claim 1.

6. The identification method according to claim 5, comprising the following steps:
(1) primer synthesis: synthesizing the KASP molecular marker Chr06:26042043 primer combination;
(2) DNA extraction: extracting genomic DNA from the Prunus persica (L.) Batsch to be identified;
(3) KASP: with the genomic DNA extracted in the step (2) as a template, conducting the KASP using the KASP molecular marker Chr06:26042043 primer combination synthesized in the step (1); and
(4) KASP product detection and analysis: detecting a genotype of an amplified product, and according to the genotype, preliminarily determining the CR level of the Prunus persica (L.) Batsch,
wherein the step (1) and the step (2) are implemented in any order.

7. An identification method for a CR level of Prunus persica (L.) Batsch, comprising using molecular marker primer combinations, wherein the molecular marker primer combinations comprise the KASP molecular marker Chr06:26042043 primer combination according to claim 1 and a KASP molecular marker Chr01:46470090 primer combination related to the CR of the Prunus persica (L.) Batsch; wherein the KASP molecular marker Chr01:46470090 primer combination comprises a third forward primer, a fourth forward primer, and a second shared reverse primer, wherein the nucleotide sequence of the third forward primer is shown in SEQ ID NO: 4, the nucleotide sequence of the fourth forward primer is shown in SEQ ID NO: 5, and the nucleotide sequence of the second shared reverse primer is shown in SEQ ID NO: 6.

8. A kit comprising one or two of the KASP molecular marker Chr06:26042043 primer combination according to claim 1 and a KASP molecular marker Chr01:46470090 primer combination related to the CR of the Prunus persica (L.) Batsch; wherein the KASP molecular marker Chr01:46470090 primer combination comprises a third forward primer, a fourth forward primer, and a second shared reverse primer, wherein the nucleotide sequence of the third forward primer is shown in SEQ ID NO: 4, the nucleotide sequence of the fourth forward primer is shown in SEQ ID NO: 5, and the nucleotide sequence of the second shared reverse primer is shown in SEQ ID NO: 6.

9. The identification method according to claim 5, wherein in the KASP molecular marker Chr06:26042043 primer combination, different fluorescent tags are added to 5′ terminus of the first forward primer and the second forward primer, respectively.

10. A identification method for a CR level of Prunus persica (L.) Batsch, comprising using the KASP molecular marker Chr01:46470090 primer combination according to claim 3.

11. The identification method according to claim 10, wherein in the KASP molecular marker Chr01:46470090 primer combination, different fluorescent tags are added to 5′ terminus of the third forward primer and the fourth forward primer, respectively.

12. The identification method according to claim 9, comprising the following steps:
(1) primer synthesis: synthesizing the KASP molecular marker Chr06:26042043 primer combination;
(2) DNA extraction: extracting genomic DNA from the Prunus persica (L.) Batsch to be identified;
(3) KASP: with the genomic DNA extracted in the step (2) as a template, conducting the KASP using the KASP molecular marker Chr06:26042043 primer combination synthesized in the step (1); and
(4) KASP product detection and analysis: detecting a genotype of an amplified product, and according to the genotype, preliminarily determining the CR level of the Prunus persica (L.) Batsch,
wherein the step (1) and the step (2) are implemented in any order.

13. The identification method according to claim 10, comprising the following steps:
(1) primer synthesis: synthesizing the KASP molecular marker Chr01:46470090 primer combination;
(2) DNA extraction: extracting genomic DNA from the Prunus persica (L.) Batsch to be identified;
(3) KASP: with the genomic DNA extracted in the step (2) as a template, conducting the KASP using the KASP molecular marker Chr01:46470090 primer combination synthesized in the step (1); and
(4) KASP product detection and analysis: detecting a genotype of an amplified product, and according to the genotype, preliminarily determining the CR level of the Prunus persica (L.) Batsch,
wherein the step (1) and the step (2) are implemented in any order.

14. The identification method according to claim 11, comprising the following steps:
(1) primer synthesis: synthesizing the KASP molecular marker Chr01:46470090 primer combination;
(2) DNA extraction: extracting genomic DNA from the Prunus persica (L.) Batsch to be identified;
(3) KASP: with the genomic DNA extracted in the step (2) as a template, conducting the KASP using the KASP molecular marker Chr01:46470090 primer combination synthesized in the step (1); and
(4) KASP product detection and analysis: detecting a genotype of an amplified product, and according to the genotype, preliminarily determining the CR level of the Prunus persica (L.) Batsch,
wherein the step (1) and the step (2) are implemented in any order.

15. The identification method according to claim 7, wherein in the KASP molecular marker Chr01:46470090 primer combination, different fluorescent tags are added to 5′ terminus of the third forward primer and the fourth forward primer, respectively.

16. The identification method according to claim 7, wherein in the KASP molecular marker Chr06:26042043 primer combination, different fluorescent tags are added to 5′ terminus of the first forward primer and the second forward primer, respectively.

17. The identification method according to claim 16, wherein in the KASP molecular marker Chr01:46470090 primer combination, different fluorescent tags are added to 5′ terminus of the third forward primer and the fourth forward primer, respectively.

18. The kit according to claim 8, wherein in the KASP molecular marker Chr01:46470090 primer combination, different fluorescent tags are added to 5′ terminus of the third forward primer and the fourth forward primer, respectively.

19. The kit according to claim 8, wherein in the KASP molecular marker Chr06:26042043 primer combination, different fluorescent tags are added to 5′ terminus of the first forward primer and the second forward primer, respectively.

20. The kit according to claim 19, wherein in the KASP molecular marker Chr01:46470090 primer combination, different fluorescent tags are added to 5′ terminus of the third forward primer and the fourth forward primer, respectively.
